(19) United States
(12) Patent Application Publication
(10) Pub. No.: US 2007/0083252 A1
(43) Pub. Date: Apr. 12, 2007

(54) Method for placing a stent through a constricted lumen, and medical device

(76) Inventor: Michael B. McDonald, Cordova, TN (US)

Correspondence Address: PETER L. BREWER BEARMAN, CALDWELL, & BERKOWITZ;LAW FIRM, 165 MADISON AVENUE, SUITE 2000, MEMPHIS, TN, 38103, US

(21) Appl. No.: 11/236,132

(22) Filed: Sep. 27, 2005

(51) Int. Cl. A61F 2/06; A61F 11/00

(52) U.S. Cl. 623/1.11; 606/108

(57) ABSTRACT

A method for advancing a stent through a constricted lumen of a patient, in which a medical device having an access tube is urged into the lumen via a slip wire. The access tube defines a tubular body having a slip wire connected thereto. The access tube is advanced into the lumen by feeding the slip wire to a point of partial blockage. The access tube is configured to radially compress or comply upon encountering a narrowed area of the lumen. A stent may then also be advanced to the point of blockage, where it enters the compliant access tube. The stent is then urged through the constricted portion of the lumen by traversing the compliant access tube. Preferably, the access tube has a slit running along its length, and is fabricated from a hydrophilic material.

### BACKGROUND OF THE INVENTION

1. Field of the Invention

The present invention relates to medical procedures and devices. More particularly, the invention relates to a device and method for advancing a stent through a constricted lumen, such as a coronary artery, in a patient.

2. Description of the Related Art

Percutaneous transluminal coronary angioplasty, also referred to as PTCA, is a well-known, non-surgical treatment used for opening blocked arteries. This procedure is sometimes referred to as balloon angioplasty or balloon dilation. Angioplasty is typically performed in a cardiac catheterization lab, or “cath lab,” by a cardiologist and cardiology team. The procedure widens, or “dilates,” blocked arteries, and can help prevent the complications of atherosclerosis.

During angioplasty, a small needle is used to first puncture the femoral artery at the level of the patient's hip. Less commonly, access may be acquired using an artery in the arm or wrist area. The punctured artery is used as the point of entry to advance the equipment used to open blockages inside a coronary artery. To aid in maintaining access through the femoral artery, an introducer sheath is placed in the femoral artery. The introducer sheath is typically a short, tubular device that extends into and out of the epidermal puncture by several inches.

Next, a guidewire is inserted through the introducer sheath. The guidewire defines a thin, elongated wire. The guidewire includes a soft, flexible tip for navigating through vessels without insulting the inner vessel wall. The distal end of the guidewire is manipulated through the arterial system to reach the ascending of the aorta.

A guide catheter is next inserted into the sheath. The guide catheter defines an elongate tube radially dimensioned to be inserted from the femoral artery into the coronary artery. The guide catheter is run over the guidewire through the sheath. The guide catheter is further passed through the femoral artery until it reaches into the aorta. The distal end of the guide catheter rests at or near the opening of the artery under treatment.

A balloon catheter is next advanced over the guidewire and through the sheath. The balloon catheter is further advanced through the guide catheter and then beyond its distal end. The balloon catheter is still further advanced over the guidewire and placed adjacent a targeted area of treatment within the coronary artery. Once positioned, the balloon catheter is fully inflated at least once, and often several times, in order to expand the balloon against the inner wall of the vessel. Expansion of the balloon causes the arterial wall to stretch, and also flattens the deposits along the wall that are causing the blockage.

More recently, an additional procedure has been developed for use in clearing arterial blockages. This procedure involves the placement of a permanent stent along the balloon. The stent defines a small, expandable tubular device that is run into the artery around the balloon. When the balloon is inflated, the balloon radially expands the stent into frictional engagement with the surrounding inner wall of the vessel. The stent props open a clogged artery to enable fuller blood flow. The stent may also include medicaments for treatment of the vessel wall to decrease the chance of reblockage and development of scar tissue. When the balloon is contracted for removal from the artery, the stent is released from the balloon and remains in place within the coronary artery. The stent provides a more permanent way to clear arterial blockages.

It has been observed that in some patients it is difficult to advance the stent much beyond the distal end of the guide catheter. In this respect, coronary arteries sometimes develop calcium or other deposits of various thicknesses along a vessel wall. While a small buildup may not call for placement of an expensive stent, such a buildup may nevertheless impede the advancement of the stent to the point of more desired treatment. Alternatively, the tortuous geometry of a coronary artery may create a restriction in the advancement of the stent.

Therefore, a need exists for a procedure for advancing the stent through the partially constricted artery. A need also exists for a medical device that facilitates the advancement of the stent through a constricted lumen in a coronary artery, or through a previously placed coronary stent.

### SUMMARY OF THE INVENTION

A method for advancing a stent through a constricted lumen of a patient is first provided. In one aspect, the method includes the step of running an access tube into the patient lumen. The access tube defines a tubular body having a distal end and a proximal end. A slip wire is connected to the access tube. The method further includes the steps of further advancing the access tube into a constricted portion of the lumen, running a stent into the lumen, advancing the stent into the proximal end of the access tube, and still further advancing the stent through the access tube, thereby providing passage of the stent through the constricted portion of the lumen.

In one embodiment, the method also comprises the steps of inserting a distal end of a coronary guidewire into the coronary artery of the patient; manipulating the guidewire so that the distal end of the guidewire is delivered beyond the targeted treatment area within the artery; and, advancing a distal end of a tubular guide catheter over the guidewire to a selected point within the patient lumen but short of the distal end of the guidewire. In this embodiment, the step of running an access tube into the patient lumen comprises placing the access tube over the guidewire and urging the slip wire through the tubular catheter, into the coronary artery, and into the constricted portion of the artery where difficulty in passing a stent was encountered.

A method for advancing a stent through a constricted artery of a human patient is also provided. The method includes the steps of inserting a guide catheter into the femoral artery of the patient; inserting the distal end of a coronary guidewire into the guide catheter; manipulating the guidewire through the guide catheter and into a coronary artery so that the distal end of the guidewire is positioned along and beyond a targeted treatment area within a coronary artery; inserting an access tube onto the guidewire, the access tube defining a tubular body having a distal end and a proximal end, with a slip wire connected to the access tube; urging the slip wire into the guide catheter so as to advance the distal end of the access tube over the guidewire and towards the distal end of the catheter; further advancing the access tube into a constricted portion of the coronary artery beyond the distal end of the catheter; placing an intravascular balloon and stent into the artery; advancing the stent through the catheter over the coronary guidewire; further advancing the stent into the proximal end of the access tube; and, still further advancing the stent through the access tube. In this way, passage of the stent through the constricted portion of the coronary artery is provided.

In one embodiment, the method further includes the steps of still further advancing the stent through the access tube; and then disposing the stent at a determined point of treatment within the coronary artery. The method may also include the steps of removing the guidewire from the femoral artery, actuating the stent so as to implant the stent at the determined point of treatment, and removing the slip wire and connected access tube from the coronary artery and the femoral artery.

Preferably, the access tube comprises an elongated tubular wall having a slit running substantially along a length of the access tube. The wall defines a bore within the access tube. The wall is fabricated from a hydrophilic material such as Nitinol™ material or the like. The access tube receives the guidewire and the stent through the bore.

In another embodiment, a method for inflating a stent in a coronary artery of a human patient is provided. The method comprises the steps of running an access tube into the coronary artery, locating the access tube along a point of treatment within the coronary artery, running the stent into the access tube to the point of treatment, withdrawing the access tube so as to expose the stent to the surrounding coronary artery, and inflating the stent so as to permanently dilate the coronary artery at the point of treatment.

A medical device for advancing a stent through a constricted lumen of a patient is also provided. In one embodiment, the device includes a slip wire having a proximal end and a distal end; and a tubular body near the distal end of the slip wire, the tubular body having a proximal opening, a distal opening, and a tubular wall defined therebetween. The wall is fabricated from a deformable and hydrophilic material. Preferably, the wall comprises a slit running substantially along its length.

### BRIEF DESCRIPTION OF THE DRAWINGS

So that the manner in which the above recited features of the present invention can be better understood, certain drawings are appended hereto. It is to be noted, however, that the appended drawings illustrate only selected embodiments of the inventions and are therefore not to be considered limiting of scope, for the inventions may admit to other equally effective embodiments and applications.

FIG. 1 is a perspective view of a medical device in accordance with the present invention, in one embodiment. The medical device includes an access tube.

FIG. 2A presents a side view of a portion of the medical device of FIG. 1. The access tube is illustrated being moved through a guide catheter within an arterial wall. The guide catheter and arterial wall are seen in cross-section.

FIG. 2B is another side view of the medical device of FIG. 1. Here, the medical device is illustrated as being further advanced beyond a distal end of the guide catheter.

FIG. 3 provides a cross-sectional of the device of FIG. 1, taken along line 3-3 of FIG. 2B.

FIG. 4A presents a side view of a stent being advanced into a coronary artery. The stent is traveling over a guidewire previously placed in the artery. The stent is illustrated about to encounter a constricted portion of the artery.

FIG. 4B demonstrates that the medical device has advanced through the coronary artery to the point of the occlusion. The stent is unable to further advance through the coronary artery without rubbing across a buildup of material interior to the coronary artery.

FIG. 5A shows that the access tube of FIG. 1 has been run into the artery and across the occlusion of FIG. 4B. In addition, the stent is being re-run into the coronary artery. The stent has entered the access tube.

FIG. 5B is a side view showing the stent having now advanced beyond the distal end of the access tube and beyond the constricted portion of the coronary artery. The stent may be further advanced to a designated point of treatment. The access tube has enabled the stent to traverse the constricted portion of the artery without injuring the artery.

### DETAILED DESCRIPTION

Definitions

As used herein, the term “patient” refers to any mammal in need of medical treatment.

The term “lumen” refers to any opening in a patient including, for example, a human artery.

The term “access artery” may be any artery used by a medical service provider such as a cardiologist or cardiology team to obtain access to an area of occlusion within a patient's arterial system. This may be, for example, an artery in the patient's arm or wrist. It may also be, for example, an artery near the patient's groin, such as the femoral artery.

The terms “constricted lumen” or “constricted portion of a lumen” mean any restriction to the passage of a stent. Non-limiting examples include buildup of material along an arterial wall, a tortuous bend in an artery (or other lumen) or a previously-placed stent.

Description of Specific Embodiments

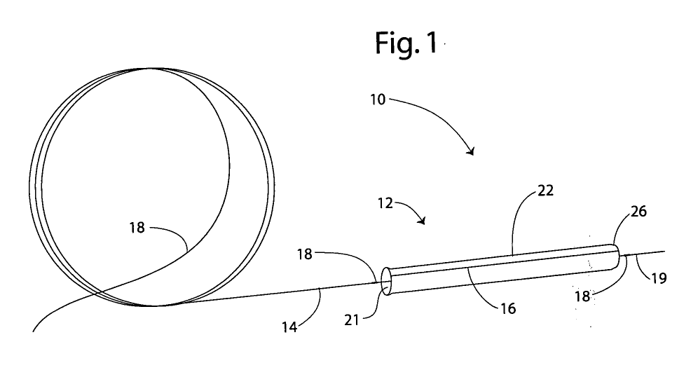

FIG. 1 presents a perspective view of a medical device 10 in accordance with the present invention, in one embodiment. The medical device 10 is designed to be advanced into a lumen (not shown in FIG. 1) of a patient. It is understood that the lumen may be any opening; however, in the present disclosure the medical device 10 and accompanying methods of use are described in a context in which the lumen is the artery of a human patient. An arterial wall of an artery 30 is shown in subsequent figures herein.

The medical device 10 includes an access tube 12 and a slip wire 14. The slip wire 14 defines a long, slender, solid member used for feeding the access tube 12 into the patient's artery 30. The slip wire 14 may be fabricated from any material that is of sufficient stiffness to allow the medical service provider to apply compression to the wire 14 in order to urge the devise 10 into the patient and through a lumen. Such materials may be, for example, a metallic material such as an alloy, or a composite material such as a polycarbonate. The slip wire 14 should also be flexible enough to allow the wire 14 to negotiate turns such as may be encountered within the patient's arterial system.

The slip wire 14 has a proximal end 18 and a distal end 19. In the perspective view of FIG. 1, the material adjacent the proximal end 18 is rolled for ease of manipulation. Optionally, the medical device 10 may include a spool (not shown) that allows the medical service provider to unreel the slip wire 14 as the device 10 is slowly fed into a patient's lumen 30.

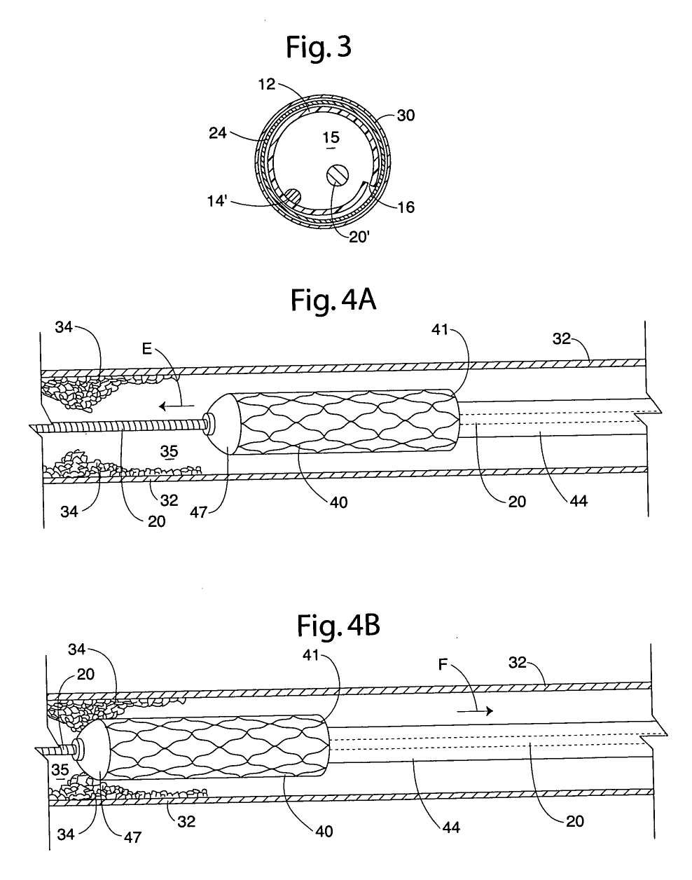

The access tube 12 of the medical device 10 comprises a tubular body 22 disposed near the distal end 19 of the slip wire 14. The access tube 12 has a proximal end 21 and a distal end 26. It is preferred that the proximal end 21 of the tube 12 be flanged outwardly, while the distal end 26 of the tube 12 be flanged inwardly. The proximal 21 end and the distal end 26 are open, and the access tube 12 defines an elongated bore 15 (as best illustrated in FIG. 3A). The proximal and distal ends 21, 26 of the tube 12 allow fluid communication through the bore 15 along the length of the access tube 12.

The access tube 12 is fabricated from a flexible material. Preferably, the access tube 12 is also fabricated from a water absorbent or hydrophilic material. In this way, as the tube 12 contacts blood or other fluids within an artery 30, the tube 12 acquires a slippery property. An example of a suitable material is Nitinol™ material.

The access tube 12 is configured to collapse when encountering a reduced inner diameter portion of a lumen. In the arrangement of FIG. 1, the access tube 12 includes a slit 16. The slit 16 runs the length of the access tube 12. The slit 16 allows the access tube 12 to radially constrict in order to accommodate reductions in the inner diameter of the artery 30 as the access tube 12 is advanced through a patient's arterial system.

It is noted here that the slip wire 14 preferably runs the length of the access tube 12 and extends beyond the access tube 12. This imbues a sufficient stiffness to the access tube 12 to allow it to withstand compressive forces exerted along the tube 12 as the medical service provider, e.g., a cardiologist, pushes the medical device 10 into the patient's body.

It is also noted that the distal end 19 of the slip wire 14 may include a softer, more compliant portion than the remainder of the slip wire 14. The distal end 19 may also include a curved tip (not shown). Such features allow the medical device 10 to be urged through an arterial system without unduly insulting or injuring the inner walls of the vessel 30.

The access tube 12 also preferably includes opposing radiopaque markers 18. One marker 18 is positioned near the proximal end 21 of the tube 12, while the other marker 18 is positioned near the distal end 26 of the tube 12. The markers 18 may be attached to the wall of the access tube 12. However, it is preferred that the markers 18 be incorporated into the slip wire 14. The radiopaque markers 18 are used by the cardiologist or other medical service provider to confirm the position of the access tube 12 within the patient.

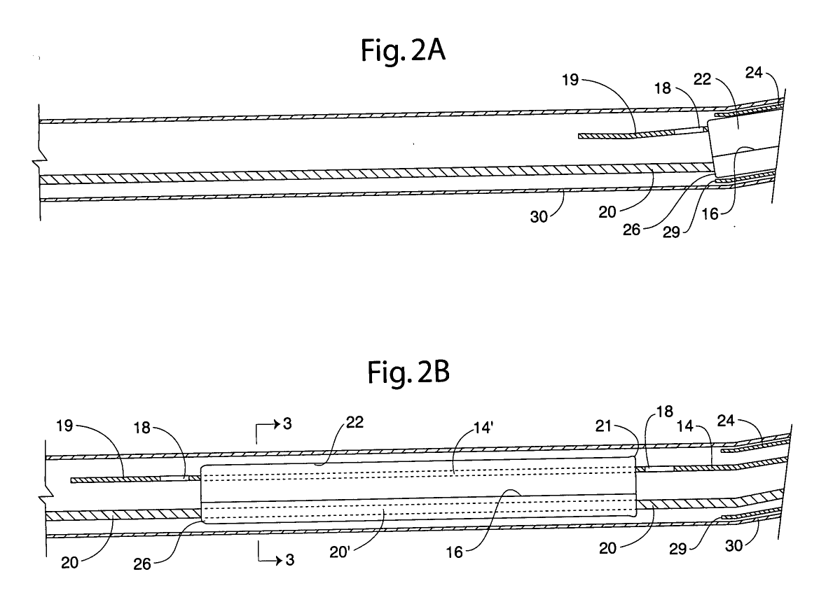

FIG. 2A presents a side view of a portion of the medical device 10 of FIG. 1. The medical device 10 has been urged into the artery 30 of a patient (not shown) in accordance with angioplasty procedures. The distal end 26 of the elongated access tube 12 is seen within the surrounding artery 30.

A portion of a guide catheter 24 is seen in FIG. 2A. As noted above, the guide catheter 24 is advanced into the patient's arterial system, including artery 30. The guide catheter 24 enables a stent (shown at 40 in FIG. 4A) to be later inserted into the patient's arterial system without frictional contact with the vessel walls, at least as to those vessel wall portions that receive the guide catheter 24. A distal end 29 of the guide catheter 24 is seen in cross-section within the surrounding arterial wall 30.

In FIG. 2A, the distal end 26 of the access tube 12 has reached the distal end 29 of the surrounding guide catheter 24. The slit 16 of the access tube 12 running to the distal 26 end is also visible in FIG. 2A. Also visible is the distal end 19 of the slip wire 14. These features are more fully seen in FIG. 2B, which follows.

FIG. 2B presents another side view of the medical device 10 of FIG. 1. Here, the device 10 has been advanced beyond the distal end 29 of the guide catheter 24. The proximal 21 and distal 26 ends of the access tube 12 are each shown. In addition, the length of the slit 16 is visible.

Certain components are also shown residing within the access tube 12. First, a guidewire 20 has been previously placed in the patient's artery 30. The guidewire 20 can be seen in FIG. 2B along the length of the access tube 12. A portion 20′ of the guidewire 20 extends through the bore (noted at 15 in FIG. 3) of the access tube 12. This portion 20′ of the guidewire 20 is shown in broken lines. As noted, the guidewire 20 serves as a guide for running various instruments into the patient's artery 30 during angioplasty, most commonly the angioplasty balloon and stent.

The slip wire 14 of the medical device 10 is also shown in FIG. 2B. The distal end 19 of the slip wire 14 extends beyond the distal end 26 of the access tube 12. That portion of the slip wire 14 residing within the access tube 12 is shown in broken lines at 14′. The slip wire 14′ is optionally connected to an inner surface along the length of the access tube 12 to provide stiffness. Connection may be by adhesive bond, thermal silicone attachment, or any other biocompatible means. One example is to mold the slip wire 14′ into the access tube 12.

FIG. 3 provides a cross-sectional view of the device 10 of FIG. 1, taken along line 3-3 of FIG. 2B. The guidewire portion 20′ can be seen within the bore 15 of the access tube 12. Similarly, the slip wire portion 14′ can be seen within the bore 15 of the access tube 12. In the embodiment of FIGS. 2B and 3, the slip wire 14′ connects to the inner wall of the access tube 12. This provides longitudinal support for the access tube 12 and facilitates advancement of the access tube 12 during angioplasty. Optionally, the slip wire 14′ may terminate at or near the proximal end 21 of the access tube 12.

The cross-sectional view of FIG. 3 also shows functionality of the slit 16. It can be seen that the slit 16 permits the access tube 12 to radially compress in response to any narrowing of the inner diameter of a surrounding artery. In this respect, the access tube 12 is deployed by a medical services or health care provider to facilitate the advancement of other medical instrumentation when the lumen is constricted. As noted above, such an example would be the advancement of a stent during angioplasty.

During balloon angioplasty, a stent is advanced into the patient's arterial system, and into a coronary artery proximate the patient's heart. FIG. 4A presents a side view of the stent 40 being advanced into a coronary artery 32. The stent 40 is traveling over the guidewire 20 previously placed in the arteries 30, 32. The stent 40 is urged into the arteries 30, 32 by applying compression to a balloon slip wire 44. The stent 40 has a proximal end 41 and a distal end 47. The stent 40 is inflated by actuation of an interior balloon (not shown).

In the view of FIG. 4A, the stent 40 is traveling through a bore 35 of the coronary artery 32. Arrow E indicates the direction of travel. It can be seen that the artery 32 includes a constricted portion 34 of the artery 32. The partial blockage 34 or buildup does not unduly restrict blood flow, but does potentially inhibit advancement of the stent 40.

In order to employ the device 10, the cardiology team will remove the stent 40 from the patient's body. FIG. 4B demonstrates that the medical device 10 has advanced through the coronary artery 32 to the point of the occlusion 34. The stent 40 is unable to further advance through the coronary artery 32 across the buildup 34. Therefore, the stent 40 is now to be removed from the artery 32. Arrow E shows the direction of the stent 40 for removal.

Upon removal of the stent 40, the cardiologist will direct that the medical device 10 be inserted into the patient lumen 30. The device 10 will then be advanced through the guide catheter 24 within the artery 30 (step shown in FIG. 2A), and then further advanced beyond the distal end 29 of the guide catheter 24 (step shown in FIG. 2B). The device 10 is still further advanced into the coronary artery 32, to the point of partial constriction (occluded portion 34 seen in FIGS. 4A and 4B).

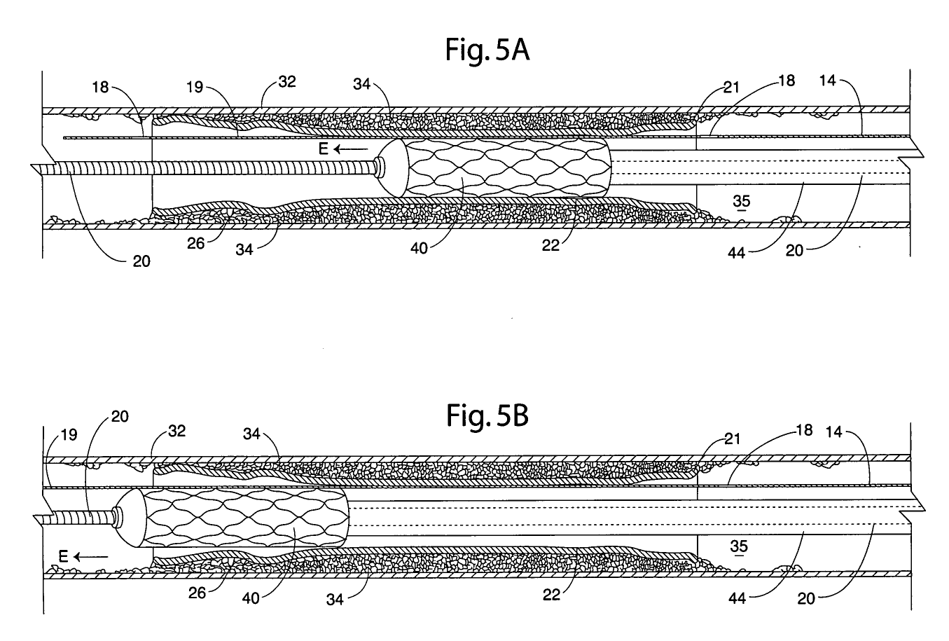

FIG. 5A shows that the medical device 10 has been run into the coronary artery 32. Compressive force has been applied to the slip wire 14 to urge the slip wire 14 into the guide catheter 24 so as to advance the access tube 12 through catheter 24. The access tube 12 has been further advanced to the constricted portion 34 of the coronary artery 32 beyond the distal end 29 of the catheter 24. In FIG. 5A, the distal end 19 of the slip wire 14 has cleared the occlusion 34 of FIG. 4B. Further, the access tube 12 has been placed across the occlusion 34.

As noted, the access tube 12 is fabricated from a hydrophilic material which has a lubricative quality as it moves through vessels. These features permit the access tube to move through the constriction 34. The compliant nature of the wall that forms the access tube 12 can be seen. In this respect, the profile of the access tube 12 at least partially conforms to the inner diameter of the surrounding artery 32. Further, the slit 16 permits the radius of the access tube 12 to more readily comply with the partial occlusion 34 presented in the artery 32. In this respect, the body or wall of the access tube 12 is able to radially constrict.

FIG. 5A also shows the step of running the stent 40 back into the coronary artery 32. Here, it can be seen that the stent 40 has progressed through the outwardly flanged proximal end 21 of the access tube 12, and is now within the bore 15 of the access tube 12. Arrow E demonstrates the direction of travel for the stent 40. The stent 40 travels over the guidewire 20 to the point of constriction 34.

FIG. 5B provides another side view showing the stent 40 having now advanced beyond the distal end 26 of the access tube 12. More importantly, the stent 40 has advanced beyond the constricted portion 34 of the coronary artery 32. The medical device 10 has enabled the stent 40 to traverse the constricted portion 34 of the artery 32 without injuring the artery 32. The stent 40 may now be further advanced to a designated point of treatment in accordance with angioplasty procedures. Those of ordinary skill in the art will understand the use of x-ray equipment and angiograms to identify points of occlusion.

The length of the access tube 12 may vary. In one arrangement, the length is short enough to traverse an anticipated section of coronary blockage. In another arrangement, the length is long enough so that the proximal end 21 of the access tube 12 remains within the guide catheter 24 while the distal end 26 extends beyond an anticipated section of coronary blockage.

It is to be noted that other variations of the use of the access tube 12 exist. For instance, the access tube 12 may be advanced within the artery bore 35 to the point of partial blockage 34 before the stent 40 is ever inserted into the patient. If it is anticipated that a second point of partial blockage will be encountered, then the stent 40 may be retained within the bore 15 of the access tube 12 and the access tube 12 then advanced along with the stent 40 therein. Once this second portion is navigated, the stent 40 may be urged beyond the access tube 12 and to the desired point of treatment. The medical device 10 may then be withdrawn from the patient. The medical device 10 may be removed before or after expansion of the stent 40.

Alternatively, the medical device 10 may be advanced within the patient's arterial system to the point of desired treatment ahead of a stent 40. Once the access tube 12 is positioned across the substantially blocked artery, the cardiologist will push the stent 40 into the access tube 12. When the stent 40 is at the point of desired treatment and ready for inflation, the access tube 12 is pulled back to expose the stent 40. In this way, the stent 40 is able to be placed within an area of treatment without injuring the surrounding artery wall. The medical device 10 is removed before expansion of the stent 40.

It is also noted that in FIGS. 4A and 4B, the illustrated occlusion 34 is caused by calcium buildup. However, other types of partial blockages may be encountered. This may arise from a previously-placed stent. Additionally, a tortuous bend in an artery may create such a restriction. The medical device 10 and methods disclosed herein have equal application to traversing such other blockages.

While this disclosure is written in the context of advancing a stent through a human patient's coronary artery, it is understood that the device and procedures have equal utility in navigating through narrow and constricted lumen of any biological type and in any mammal.

I claim:

1. A method for advancing a stent through a constricted lumen of a patient, comprising the steps of:
running an access tube into the patient lumen, said access tube defining a tubular body having a distal end and a proximal end, with a slip wire connected to the access tube;
further advancing the access tube into a constricted portion of the patient lumen;
advancing a stent in the patient lumen to the proximal end of the access tube;
further advancing the stent into an inner bore of the access tube; and
still further advancing the stent through the bore and beyond the distal end of the access tube and the constricted portion of the lumen.

2. The method of claim 1, further comprising the steps of:
inserting a guide catheter into the patient lumen;
inserting a coronary guidewire into the guide catheter;
manipulating the guidewire so that a distal end of the guidewire is delivered to a targeted treatment area within the lumen and beyond a distal end of the guide catheter.

3. The method of claim 2, wherein the step of running an access tube into the patient lumen comprises placing the access tube over the guidewire and urging the slip wire into the guide catheter.

4. The method of claim 3, wherein the step of advancing the stent further comprises advancing the stent through the guide catheter to the distal end of the catheter, and then on to the location of the access tube.

5. The method of claim 4, wherein:
the constricted portion of the lumen is beyond the distal end of the guide catheter; and
the step of further advancing the access tube into a constricted portion of the lumen comprises advancing the distal end of the access tube over the guidewire and beyond the distal end of the catheter.

6. The method of claim 2, further comprising the steps of:
still further advancing the stent in the patient's lumen; and
disposing the stent at a determined point of treatment within the lumen beyond the access tube.

7. The method of claim 6, wherein the lumen is an artery, and the method further comprises the steps of:
actuating the stent so as to implant the stent at the determined point of treatment within the artery; and
removing the guidewire from the artery.

8. The method of claim 6, further comprising the step of:
removing the slip wire and connected access tube from the lumen.

9. The method of claim 1, wherein the access tube comprises an elongated, radially deformable body.

10. The method of claim 9, wherein the access tube further comprises a slit running substantially along a length of the access tube.

11. The method of claim 9, wherein the access tube is fabricated from a hydrophilic material.

12. The method of claim 11, wherein the access tube is fabricated from Nitinol material.

13. The method of claim 1, wherein the patient is a human.

14. The method of claim 5, wherein:
the lumen is a coronary artery; and
the constriction in the constricted portion of the lumen is caused by any of a pre-existing stent, a calcium buildup or the anatomy of the artery.

15. A method for advancing a stent through a constricted artery of a human patient, comprising the steps of:
inserting a guide catheter into an access artery of the patient;
advancing a distal end of the guide catheter to a selected point within the coronary artery;
running a coronary guidewire into the guide catheter;
advancing a distal end of the guidewire beyond the distal end of the guide catheter at least to the constricted artery;
running an access tube into the guide catheter and over the guidewire, the access tube defining a tubular body having a distal end and a proximal end, with a slip wire connected to the access tube;
urging the slip wire into the catheter so as to advance the distal end of the access tube over the coronary guidewire and towards the distal end of the guide catheter;
further advancing the access tube into a targeted treatment area of the coronary artery beyond the distal end of the catheter;
placing an intravascular stent over the coronary guidewire;
advancing the stent through the guide catheter to the distal end of the catheter;
further advancing the stent into the proximal end of the access tube; and
still further advancing the stent through the access tube, thereby providing passage of the stent through the constricted portion of the coronary artery.

16. The method of claim 15, further comprising the steps of:
still further advancing the stent along the coronary artery; and
disposing the stent at a targeted treatment area within the coronary artery.

17. The method of claim 16, further comprising the steps of:
retracting the slip wire and connected access tube from the access artery; and
actuating the stent so as to implant the stent at the targeted treatment area.

18. The method of claim 15, wherein the access tube comprises an elongated tubular body having a slit running substantially along a length of the access tube, and the body being fabricated from a hydrophilic material

19. The method of claim 18, wherein the body is open at opposing ends to define a bore within the access tube.

20. The method of claim 19, wherein the body of the access tube is fabricated from Nitinol material.

21. A medical device for advancing a stent through a constricted lumen of a patient, comprising:
a slip wire having a proximal end and a distal end; and
a tubular body near the distal end of the slip wire, the tubular body having a proximal opening, a distal opening, and a tubular wall defined therebetween, the wall being fabricated from a deformable and hydrophilic material.

22. The medical device of claim 21, wherein the body comprises a slit running substantially along its length.

23. The medical device of claim 21, further comprising a first radiopaque marker near the proximal opening, and a second radiopaque marker near the distal opening.

24. A method for inflating a stent in a coronary artery of a human patient, comprising the steps of:
running an access tube into the coronary artery, said access tube defining a tubular body having a distal end and a proximal end, with a slip wire connected to the tubular body;
positioning the access tube along a point of treatment within the coronary artery;
running the stent into the access tube to the point of treatment;
withdrawing the access tube so as to expose the stent to the surrounding coronary artery at the point of treatment; and
inflating the stent so as to dilate the coronary artery at the point of treatment.
